(19) United States
(12) Patent Application Publication
(10) Pub. No.: US 2025/0345103 A1
(43) Pub. Date: Nov. 13, 2025

(54) DEVICE FOR TREATING A BONE FRACTURE

(71) Applicant: Wolfgang Auf, Graz (AT)

(72) Inventor: Wolfgang Auf, Graz (AT)

(21) Appl. No.: 18/729,725

(22) PCT Filed: Nov. 7, 2022
(86) PCT No.: PCT/AT2022/060380

§ 371 Date: Dec. 31, 2024

(51) Int. Cl. A61B 17/72

(52) U.S. Cl. CPC A61B 17/7233

(57) ABSTRACT

The invention relates to an apparatus for treating a fracture of a bone, in particular a human long bone, comprising a bone nail (1) having a first end (2) and a second end (3), with at least one first bore (4) in a region of the first end (2) and at least one second bore (5) in a region of a second end (3) being provided transversely to a longitudinal axis (6) of the bone nail (1), so that the bone nail (1) can be fixed in place in the bone by locking pins (7) arranged in the bores (4, 5) and protruding out of the bores (4, 5). In order to enable a stable positioning in a bone in a simple manner, it is provided according to the invention that the bone nail (1) comprises a channel (8) that extends, at least in regions, approximately along a longitudinal axis (6) in the bone nail (1) and, on one side, is connected to the at least one second bore (5) and, on the other side, to an introduction opening (9) arranged in a region between the first end (2) and the second bore (5) in a lateral surface (19) of the bone nail (1), so that an illumination of the second bore (5) is possible from an interior of the bone nail (1) in that a lamp (10) is inserted into the channel (8) via the introduction opening (9) and is moved into a region of the second bore (5) in order to be able to determine a position of the second bore (5) with the aid of an emission of light from the second bore (5) when the bone nail (1) is arranged in a bone.

The invention relates to an apparatus for treating a fracture of a bone, in particular a human long bone, comprising a bone nail having a first end and a second end, with at least one first bore in a region of the first end and at least one second bore in a region of the second end being provided transversely to a longitudinal axis of the bone nail, so that the bone nail can be fixed in place in the bone by locking pins arranged in the bores and protruding out of the bores.

From the prior art, apparatuses of the type named at the outset, which are referred to as bone nails, have become known in order to connect the individual bone parts and to thus expedite or enable a healing, in particular in the case of fractures of human long bones, such as a fracture of the femur, for example.

For this purpose, the bone nail is introduced into the bone along a longitudinal axis of the bone, possibly along a bore introduced beforehand, so that the fragments of the femur fractured transversely to the longitudinal axis are connected by the bone nail. In order to prevent the bone nail from coming lose from the bone, bores are frequently provided in the bone nail that run transversely to the longitudinal axis of the bone nail and in which locking pins, in particular locking screws, can be positioned, which, if necessary, are screwed into the bone nail through a skin incision and the bone in order to connect the bone nail to the bone in a form fit such that a movement of the bone nail relative to the bone along the longitudinal axis is also blocked. The locking pins can, at an opposite end of the bore, which can be embodied as a through bore, also protrude out of the bone nail again and thus connect the bone nail to the bone on two sides. As a result, a stable positioning of the bone parts relative to one another can be ensured, whereby a healing is facilitated.

The bone nail is typically introduced into the femur from a region near the hip joint up to a region near the knee, and thus has a length that nearly equals a length of the femur.

The locking pins are typically introduced into end bores of the bone nail so that said pins are arranged in a region close to the hip and a region close to the knee or, generally speaking, at the ends of the fractured bone, in order to ensure a particularly good and stable connection between the bone nail on the one hand and the bone parts on the other hand.

When a bone nail is arranged in a femur, for example, an introduction of the locking pin into the bore near the hip, which can also be embodied as a through bore perpendicular to the longitudinal axis, is thereby typically easily possible, especially since a position of said bore on the bone nail is visible until the end, and can thus be easily determined after a complete introduction of the bone nail into the bone. For this purpose, a short aiming arm can be provided at the proximal end of the bone nail, for example, which aiming arm marks a position of the bore near the hip. Because a deformation of the bone nail in the region near the connection can be ignored, the bore near the hip is thus easily locatable for a surgeon from outside of the bone, so that the surgeon can place a bore in the bone in the correct position, through which bore the locking pin can be inserted into the bore near the hip in the bone nail in order to couple the bone nail to the bone near the hip.

However, it is difficult to precisely introduce the locking pin into the bore at that end of the bone nail that is opposite from the bore opening via which the bone nail is introduced into the bone. It can thus certainly happen that the bone nail deforms while being introduced into the bore in the bone, so that a precise determination of a position of the bore at this end, normally the distal end on a femur, is not easily possible. In particular, the determination of this distal position using a long aiming arm that extends from the proximal end outside of the bone into the region of the distal bore is liable to be highly uncertain, or is not possible in an exact manner, especially since the bone nail can be deformed by being introduced into the bone, so that a position of the distal bore changes relative to the aiming arm, which remains outside of the bone and is therefore not subject to any deformation. The determination of this position is, however, critical to the ability to position the locking pin in the bore through the bone correctly and simultaneously with minimal tissue damage.

To determine a position of this bore, it is known from the prior art to embody the bone nail with a channel extending from a face to the bore at the opposite end of the bone nail, and to introduce a powerful lamp into said channel via a bore opening arranged in the face, which lamp is moved to the bore at the opposite end of the bone nail so that, through an emission of light via the bore in which the locking pin is to be positioned and through the bone surrounding said bore, a position of the bore can be determined. An apparatus of this type is known, for example, from the document JP 2009 536 086 A.

A disadvantage in apparatuses of this type is, however, that the bone nail cannot yet be fixed in place in the bone via a locking pin positionable at an opposite end when the lamp is introduced into the bone nail, especially since a locking pin located within the bore near the face would block the channel for the lamp. With this method it is therefore merely possible to fix the bone nail in place in the bone at an end opposite from the bore opening, but a fixing by means of a locking pin in the region of the bore opening is only possible after the lamp has been removed. However, it has been shown that, as a result, an optimal positioning of the bone nail in the bone cannot be achieved in many cases.

This is addressed by the invention. The object of the invention is to specify an apparatus of the type named at the outset with which a particularly precise positioning of the bone nail in a bone is enabled, in particular in a femur of a human.

According to the invention, this object is attained by an apparatus of the type named at the outset in which the bone nail comprises a channel that extends, at least in regions, approximately along a longitudinal axis in the bone nail and, on one side, is connected to the at least one second bore and, on the other side, to an introduction opening arranged in a region between the first end and the second bore in a lateral surface of the bone nail, so that an illumination of the second bore is possible from an interior of the bone nail in that a lamp is inserted into the channel via the introduction opening and is moved into a region of the second bore in order to be able to determine a position of the second bore with the aid of an emission of light from the second bore when the bone nail is arranged in a bone.

In the course of the invention, it was recognized that an improved fixing of the bone nail in place in the bone is possible if, already before a removal of the lamp from the channel, the bone nail is locked by means of the locking pin at the first end, starting from the hip joint when the bone nail is arranged in a femur, that is, at a proximal end, before the lamp is removed from the channel and a further locking pin is arranged in a bore in a region of the distal end. According to the invention, this is possible because, in a bone nail embodied according to the invention, the lamp is no longer moved through a channel to the distal end, which channel is closed by a locking pin located in the first bore, but rather the channel opens at an introduction opening arranged in a lateral surface of the bone nail, through which opening the lamp can be inserted into the channel past a locking pin located in the first bore. As a result, a relative movement between the bone and bone nail during a removal of the lamp is eliminated, especially since the lamp is not removed from the bone nail until the bone nail has been fixed in place both at the proximal end and at the distal end, or the second end, by means of a corresponding locking pin.

An apparatus according to the invention can, in principle, be used for treating fractures in any bones, but is preferably used for fractures of long bones such as the femur, the humerus, or the tibia.

It can be provided that the introduction opening is formed by the first bore. A corresponding lamp can then be introduced via the first bore into the channel through which the lamp can be moved to the second bore, or the distal end of the bone nail, in order to illuminate the second bore from within the bone nail. The introduction opening can thus also be used as a locking bore.

If the introduction opening is formed by the first bore, it is preferably provided that a locking pin, in particular a femoral neck screw, is provided which comprises an opening such that the lamp can be inserted into the channel through the locking pin and through the first bore. In this embodiment, the first bore can thus be embodied in a conventional manner and an introduction of the lamp through the introduction opening into the first channel is achieved through the opening in the locking pin that is positioned in the first bore. The opening can be embodied as a slot, for example.

A collision of the lamp with the locking pin located in the first bore can be avoided in a particularly simple manner if the introduction opening is arranged between the first bore and the second bore on a lateral surface of the bone nail. It shall be understood that the introduction opening and the first bore can be located at different angular positions along a circumferential direction of the bone nail, which is typically embodied to be approximately cylindrical.

To be able to introduce the locking pin into the second bore with minimal injury to a tissue, an aiming arm that can be detachably connected to the bone nail is preferably provided, which aiming arm comprises a marking in a position corresponding to the second bore and can be affixed to the bone nail such that the aiming arm remains outside of the bone when the bone nail is introduced into a bone, in particular into a human femur, along a longitudinal axis in order to indicate the position of the second bore outside of the bone. Thus, by means of the aiming arm, a position outside of the bone or outside of the human body is marked, at which position the second bore would be located in the bone if the bone nail were not subject to any deformation when introduced into the bone. As a result, the tissue in this region can be opened, whereupon a precise affixing of the locking pin in the second bore can take place by means of the light emerging from the second bore and passing through the bone. The aiming arm can be removed from the bone nail once the bone nail has been positioned in the bone and fixed in place by means of the locking pin, so that only the bone nail, along with the locking pin, remains in the bone or in the human body.

It is preferably provided that the channel has, at least in regions, preferably continuously, a non-circular, in particular approximately elliptical, cross section. With a correspondingly embodied lamp, an angular position at which the light is emitted from the lamp can then be coordinated in a particularly simple manner with a corresponding angular position of the second bore and, as a result, an undesired skewing of the lamp in the channel can be reliably prevented if the lamp is embodied correspondingly. Thus, a lamp can be used in which all light is emitted at a single angular position so that a particularly high light intensity through the second bore can be achieved in order to be able to locate the second bore with particular ease.

Additionally, an instrument can be provided which corresponds to the bone nail and has a cross section that corresponds to the channel and/or the lamp, so that said instrument also creates a form fit with the channel and/or the lamp.

The channel can, in principle, run from the introduction opening to the second bore along a straight line. In order to ensure a particularly high stability of the bone nail, however, it is preferably provided that the channel comprises a first section which extends along a direction transverse to the longitudinal axis. In this manner, the channel can run to a large extent approximately along a center axis of the bone nail, whereby a minimal degradation of strength and rigidity of the bone nail by the channel is achieved. For example, the channel can run essentially centrally along a longitudinal axis of the bone nail, and can comprise a first section, aligned approximately perpendicularly to the longitudinal axis, only in the region of the introduction opening, through which section a connection of the lateral surface or the introduction opening to the center axis of the bone nail is achieved.

It is therefore preferably provided that the first section connects the introduction opening to the inner, second section of the channel, which second section approximately runs along the longitudinal axis.

To minimize a mechanical load on the lamp and nevertheless achieve a high strength and rigidity of the bone nail, it is preferably provided that the channel is curved, at least in regions. Sharp edges in the channel are then avoided, which would cause a high mechanical load for a lamp that has been introduced.

Preferably, a lamp is provided which can be introduced into the channel. It shall be understood that a corresponding lamp typically has a cross section corresponding to the channel. Particularly if the cross section of the channel is embodied to be non-circular, the corresponding lamp preferably also has a correspondingly non-circular, in particular an elliptical, cross section. The lamp can, in principle, be embodied in any desired manner that is suitable for emitting light via the second bore, or via a bore at the distal end of the bone nail. For example, the lamp can comprise one or more light-emitting diodes arranged on one side of a flexible wire, which diodes are guided into the region of the second bore by means of the wire.

Particularly preferably, it is provided that the lamp comprises a light guide. Light guides of this type are, on the one hand, flexible so that they can also be inserted into a curved channel, or into a channel with sections having different orientation. On the other hand, a strong light intensity from one or more diodes arranged at an end can be transported to a point positioned far away via corresponding light guides. If the lamp comprises a light guide, diodes that act on the light guide are typically located at a first end of the light guide and a second end of the light guide is inserted into the channel via the introduction opening in order to illuminate or light up the second bore from an interior of the bone nail.

It is thus preferably provided that the lamp is flexible so that it can be introduced without damage into the channel comprising one or more curves. Of course, alternatively to a light guide there exist additional possibilities for embodying the lamp to be flexible, for example, using a flexible probe which comprises a light-emitting diode or the like at an end.

Typically, the lamp is embodied to emit light at an end region that can be inserted into the channel.

Particularly preferably, it is provided that the lamp comprises at an end at least one lens, wherein the lens is preferably embodied as a converging lens. A particularly high light intensity can then be achieved, especially since the light emitted by the lamp is no longer emitted uniformly in all directions, but rather is gathered or focused by means of the lens.

It is preferably provided that the lamp is embodied such that, through the lens, light can be emitted along a light emission direction, which light emission direction is aligned at an angle to the longitudinal axis, in particular at an angle of approximately 90 degrees, when the lamp is arranged in the channel. It can thus be ensured that any light emitted by the lamp directly penetrates into the second bore from inside or from the channel without being absorbed or reflected by an inner wall of the channel. A correct positioning of the lamp about the longitudinal axis or along a circumferential direction can, for example, be easily ensured by a non-circular embodiment of the channel on the one hand and of the lamp on the other hand.

It is preferably provided that the channel and the lamp are embodied such that, when the lamp is inserted into the channel up to the second bore, a rotational position of the lamp relative to the channel is defined by a form fit between the lamp and channel. It can thus be ensured that not only can a position of the lamp relative to the bone nail be clearly defined in a longitudinal direction, which position can be easily determined via a length of the lamp inserted into the channel, but rather also a position of the lamp relative to the bone nail along the circumferential direction. A corresponding form fit can, for example, be achieved by a cross section of the channel and lamp that is non-circular in regions or continuously.

Additional features, advantages, and effects of the invention follow from the exemplary embodiments described below. In the drawings which are thereby referenced:

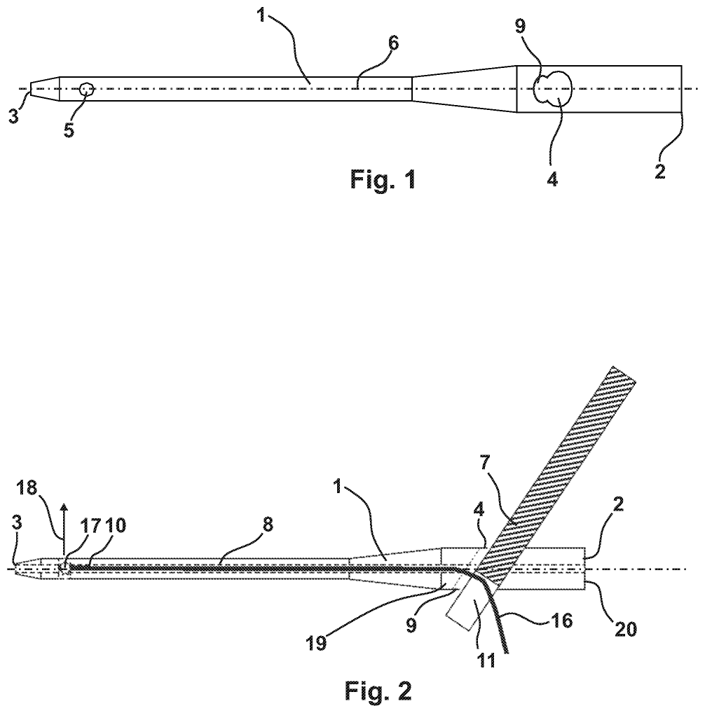

FIGS. 1 and 2 show a first exemplary embodiment of an apparatus according to the invention;

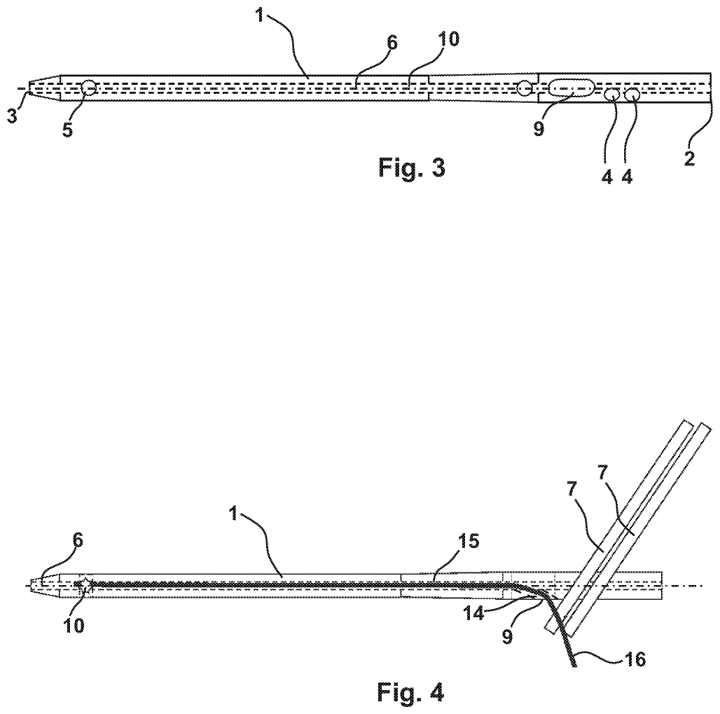

FIGS. 3 and 4 show a further exemplary embodiment of an apparatus according to the invention;

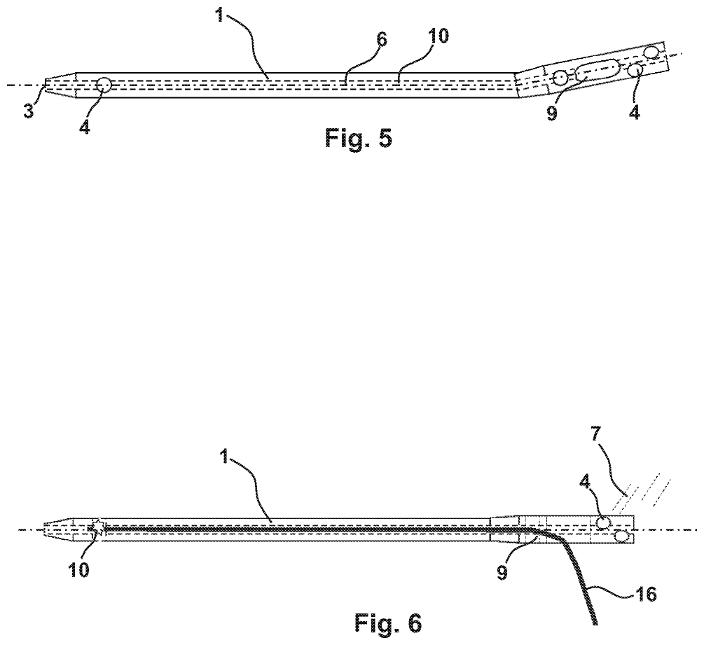

FIGS. 5 and 6 show a further exemplary embodiment of an apparatus according to the invention;

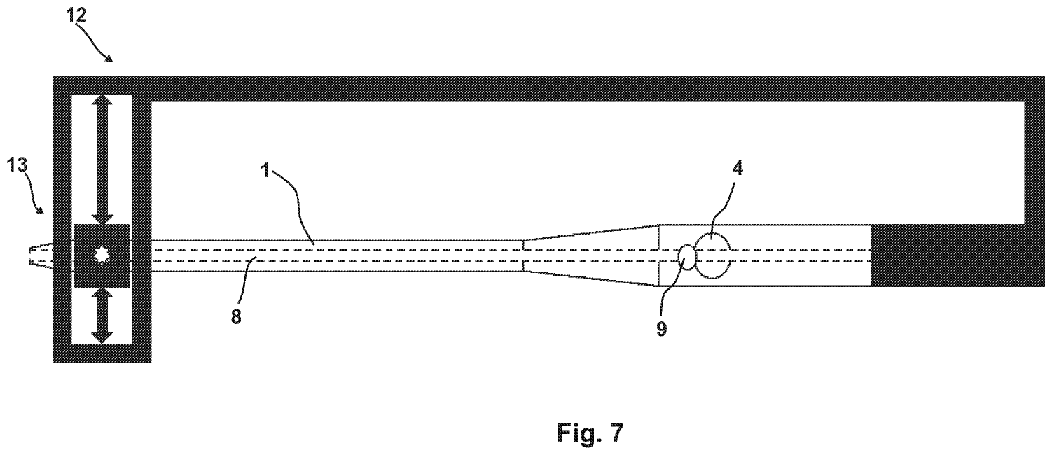

FIG. 7 shows a further exemplary embodiment of an apparatus according to the invention;

FIGS. 1 and 2 show a proximal femur nail that is embodied according to the invention, wherein FIG. 1 shows a top view and FIG. 2 shows a side view. The bone nail 1 has a longitudinal axis 6 and an essentially section-wise cylindrical contour having a peripheral lateral surface 19, a first end 2, and a second end 3, wherein the first end 2 and second end 3 are in this case formed by faces. In FIG. 1, a first bore 4 and a second bore 5 can be seen, wherein the first bore 4 is arranged in a region of a first end 2 of the bone nail 1 and the second bore 5 is arranged in a region of the second end 3 of the bone nail 1 in order to fix the bone nail 1 in place in the bone using locking pins 7 introduced into the bores 4, 5. In FIG. 2, a femoral neck screw is illustrated as an example of a locking pin 7.

This femoral neck screw can be easily positioned in the first bore 4 through the bone, for example using a short aiming arm 12 that is attached to the proximal first end 2 of the bone nail 1, especially since a deformation of the bone nail 1 in the proximal region, or a region near the connection, can be ignored.

As can be seen, the bone nail 1 comprises an inner channel 8 which, in this case, is connected to the first bore 4 on one side and to the second bore 5 on the other side. The femoral neck screw thereby comprises an opening 11 embodied as a slot, such that a lamp 10 embodied as a light guide 16 can be introduced into the channel 8 through said opening 11 in order to illuminate the second bore 5 from an interior of the bone nail 1 by means of the lamp 10. Thus, in contrast to bone nails from the prior art, the lamp 10 is in this case not introduced into the channel 8 via a face, but rather via an introduction opening 9 arranged on the lateral surface 19, whereby the lamp 10 cannot collide with a locking pin 7 arranged in the first bore 4.

Of course, the channel 8 can, as illustrated, also still extend to the face 20 in order to ensure an ease of production.

It is thus ensured that the second bore 5 can be easily located from outside of the human body with the aid of the point of light in order to fix the bone nail 1 in place by means of an additional locking pin 7 positioned in the second bore 5. The light guide 16 can then be removed again from the bone nail 1.

The lamp 10 thereby comprises a lens 17 at an end which is arranged at the end of the flexible light guide 16 such that, by means of the lens 17 embodied as a converging lens, light can be emitted in a light emission direction 18 oriented approximately perpendicularly to the longitudinal axis 6 in order to introduce light into the second bore 5 in a targeted manner. An inadvertent rotation of the lamp 10 relative to the bone nail 1 is thereby prevented by a form fit between the lamp 10 and channel 8, typically by a non-circular cross section of the channel 8 and a correspondingly non-circular cross section of the lamp 10, so that a targeted illumination of the second bore 5 is possible by means of the lens 17.

FIGS. 3 and 4 show an apparatus according to the invention, embodied as an antegrade femur nail. Here, the lamp 10 likewise embodied as a light guide 16 is not introduced into the channel 8 via the first bore 4, in which a locking pin 7 can also be positioned, but rather the introduction opening 9, also arranged here on a lateral surface 19 of the bone nail 1, is embodied separately and arranged between the first bore 4 and the second bore 5. As can be seen in FIGS. 3 and 4, multiple first bores 4 can also be arranged at the first end 2 in order to accommodate corresponding locking pins 7. The first bores 4 and the second bores 5 are typically embodied as through bores.

As illustrated here, the channel 8 comprises multiple sections 14, 15 having different orientations. For example, a first section 14 leads from the introduction opening 9 transversely to the longitudinal axis 6 into an interior of the bone nail 1, or to a center axis at which the first section 14 opens into a second section 15 of the channel 8, which is oriented along the longitudinal direction and runs centrally in the bone nail 1.

FIGS. 5 and 6 show a further apparatus according to the invention, wherein the bone nail 1 is embodied as a tibia nail in this case. Here, again, the lamp 10 is introduced into the channel 8 via an introduction opening 9 that is arranged between the first bore 4 and the second bore 5.

In all exemplary embodiments, the channel 8 can be embodied with a non-circular, preferably an elliptical, cross section, which cross section the lamp 10 can also have, at least in regions, in order to be able to easily determine a rotational position of the lamp 10 relative to the bone nail 1. The lamp 10 or the light guide 16 can also comprise a lens 17 at an end, so that an emission of light is only enabled at one or two positions in a circumferential direction, and a high light intensity is thus ensured at said positions in order to be able to easily locate the second bore 5.

FIG. 7 shows a further apparatus according to the invention, wherein an aiming arm 12 embodied as a distal aiming device is provided, which aiming arm 12 is detachably connected to the bone nail 1. In this manner, a position of the second bore 5 can already be determined before a tissue is opened in order to introduce the locking pin 7 into the second bore 5 through the bone. A locating of the second bore 5 is thus simplified and an incision kept small. As can be seen, the aiming device comprises an arm which comprises a marking 13 at a position at which the second bore 5 is located. That this position does not exactly coincide with the second bore 5 when the bone nail 1 is located in the bone can be explained by a deformation of the bone nail 1 during introduction. Therefore, for the precise locating of the second bore 5, an inner channel 8 is also provided in the bone nail 1 in this case, through which channel 8 the second bore 5 can be illuminated from an interior by means of a lamp 10.

Put differently, a position of the second bore 5 is roughly determined using the aiming arm 12 in order to make an incision in the skin at the corresponding position, whereupon an exact position of the second bore 5 is determined through an illumination from inside with the aid of a point of light on the bone in order to introduce the locking pin 7 accordingly. It shall be understood that the apparatus according to the invention can, in principle, also be used without the aiming arm 12. Particularly where an especially powerful light source is used, which in this case can also be introduced into the channel 8 through the introduction opening 9, a position of the second bore 5 can easily be determined through a layer of skin even without the aiming arm 12.

An apparatus according to the invention enables a particularly certain locating of a distal bore, wherein the bone nail 1 can still be proximally locked to the bone before a distal fixing, namely by means of a locking pin 7 that does not hinder the introduction of a lamp 10 into the channel 8. As a result, a considerably more precise positioning of the bone nail 1 in the bone is possible, and unintended relative movements during a removal of the lamp 10 can be reliably avoided. Furthermore, incisions are kept small so that a more rapid recovery after an operation is enabled.

1. An apparatus for treating a fracture of a bone, in particular of a human long bone, comprising a bone nail (1) having a first end (2) and a second end (3), with at least one first bore (4) in a region of the first end (2) and at least one second bore (5) in a region of the second end (3) being provided transversely to a longitudinal axis (6) of the bone nail (1), so that the bone nail (1) can be fixed in place in the bone by locking pins (7) arranged in the bores (4, 5) and protruding out of the bores (4, 5), characterized in that the bone nail (1) comprises a channel (8) that extends, at least in regions, approximately along a longitudinal axis (6) in the bone nail (1) and, on one side, is connected to the at least one second bore (5) and, on the other side, to an introduction opening (9) arranged in a region between the first end (2) and the second bore (5) in a lateral surface (19) of the bone nail (1), so that an illumination of the second bore (5) is possible from an interior of the bone nail (1) in that a lamp (10) is inserted into the channel (8) via the introduction opening (9) and is moved into a region of the second bore (5) in order to be able to determine a position of the second bore (5) with the aid of an emission of light from the second bore (5) when the bone nail (1) is arranged in a bone.

2. The apparatus according to claim 1, characterized in that the introduction opening (9) is formed by the first bore (4).

3. The apparatus according to claim 2, characterized in that a locking pin (7), in particular a femoral neck screw, is provided which comprises an opening (11) such that the lamp (10) can be inserted into the channel (8) through the locking pin (7) and through the first bore (4).

4. The apparatus according to claim 1, characterized in that the introduction opening (9) is arranged between the first bore (4) and the second bore (5) on a lateral surface (19) of the bone nail (1).

5. The apparatus according to one of claims 1 through 4, characterized in that an aiming arm (12) that can be detachably connected to the bone nail (1) is provided, which aiming arm (12) comprises a marking (13) in a position corresponding to the second bore (5) and can be affixed to the bone nail (1) such that the aiming arm (12) remains outside of the bone when the bone nail (1) is introduced into a bone, in particular into a human femur, along the longitudinal axis (6) in order to indicate the position of the second bore (5) outside of the bone.

6. The apparatus according to one of claims 1 through 5, characterized in that the channel (8) has, at least in regions, preferably continuously, a non-circular cross section, in particular an approximately elliptical cross section.

7. The apparatus according to one of claims 1 through 6, characterized in that the channel (8) comprises a first section (14) which extends along a direction transverse to the longitudinal axis (6).

8. The apparatus according to claim 7, characterized in that the first section (14) connects the introduction opening (9) to an inner, second section (15) of the channel (8), which second section (15) runs approximately along the longitudinal axis (6).

9. The apparatus according to one of claims 1 through 8, characterized in that the channel (8) is curved, at least in regions.

10. The apparatus according to one of claims 1 through 9, characterized in that a lamp (10) is provided which can be introduced into the channel (8).

11. The apparatus according to claim 10, characterized in that the lamp (10) comprises a light guide (16).

12. The apparatus according to claim 10 or 11, characterized in that the lamp (10) is flexible so that it can be introduced without damage into the channel (8) comprising one or more curves.

13. The apparatus according to one of claims 10 through 12, characterized in that the lamp (10) is embodied to emit light at an end region that can be inserted into the channel (8).

14. The apparatus according to one of claims 10 through 13, characterized in that the lamp (10) comprises at an end at least one lens (17), wherein the lens (17) is preferably embodied as a converging lens.

15. The apparatus according to claim 14, characterized in that the lamp (10) is embodied such that, through the lens (17), light can be emitted along a light emission direction (18), which light emission direction (18) is aligned at an angle to the longitudinal axis (6), in particular at an angle of approximately 90 degrees, when the lamp (10) is arranged in the channel (8).

16. The apparatus according to one of claims 10 through 15, characterized in that the channel (8) and the lamp (10) are embodied such that, when the lamp (10) is inserted into the channel (8) up to the second bore (5), a rotational position of the lamp (10) relative to the channel (8) is defined by a form fit between the lamp (10) and channel (8).
